(19) United States
(12) Patent Application Publication
(10) Pub. No.: US 2025/0121219 A1
(43) Pub. Date: Apr. 17, 2025

(54) NON-IMAGING TFUS SYSTEMS

(71) Applicant: Sanmai Technologies, PBC, Sunnyvale, CA (US)

(72) Inventors: Christopher DAFT, Tucson, AZ (US); Sarp SATIR, Oakland, CA (US)

(21) Appl. No.: 18/916,489

(22) Filed: Oct. 15, 2024

Related U.S. Application Data
(60) Provisional application No. 63/590,716, filed on Oct. 16, 2023.

(51) Int. Cl. A61N 7/02; A61N 7/00

(52) U.S. Cl. CPC A61N 7/02; A61N 2007/0078

(57) ABSTRACT

Systems and methods using non-imaging transcranial focused ultrasound (tFUS) systems are described. Non-imaging annular and matrix probes with low element counts are used in the systems. In an embodiment, an infrared-based system is used to gather the ultrasound's position information on the scalp. The position information is used to simulate the spatial distribution of the ultrasound field of the ultrasound probes. The simulation output is overlaid with pre-procedure MRI data and displayed to a clinician. Measurements on the MRI data are used to compensate the beam formation for the acoustic behavior of the skull so that a desirable tFUS focal region is achieved. In a different embodiment, an optical system is used for gathering positional information.

### CROSS-REFERENCE TO RELATED APPLICATIONS

This application claims priority benefit to U.S. Provisional Application No. 63/590,716 entitled “NON-IMAGING TFUS SYSTEMS”, filed Oct. 16, 2023. The subject matter of this related application is hereby incorporated herein by reference.

### TECHNICAL FIELD

This invention relates to transcranial focused ultrasound systems (tFUS), mainly to improve the focus and efficacy of the tFUS system and provide a treatment plan.

### BACKGROUND

tFUS systems help treat several types of mental illness, but the systems do not guarantee that the ultrasound stimulation accurately reaches the anatomical targets. The skull's curvature or variation in its thickness complicates the ultrasound (US) delivery to the desired location, especially with small and deep targets such as the amygdala. Substantial losses of efficacy in tFUS derive from the acoustic beam partly or entirely missing the target. Beyond the loss of efficacy, this may create unwanted side effects.

High channel count array systems can solve this problem by imaging brain anatomy and using that information to guide the stimulation. They must operate at frequencies where the acoustic wavelength is short enough to resolve brain structures; the element dimensions must be small enough in wavelengths to allow for steering, and the aperture must be large enough to create an effective focus. Such probes require sophisticated, miniaturized electronics and advanced, costly manufacturing techniques, particularly for volume guidance. Such devices require innovations in probe and silicon technology. They also require skull aberration to be controlled at higher frequencies than are needed for stimulation. The difficulty of implementing aberration compensation rises sharply with increasing frequency.

There is a need for tFUS systems that use simple, low-frequency designs to reliably deliver US stimulation to the targeted brain anatomy without the complexity of ultrasonic guidance. It would be ideal if such a tFUS system also accurately illustrates the spatial distribution of the US stimulation to a clinician.

### BRIEF DESCRIPTION OF THE DRAWINGS

FIG. 1A is an exemplary high-efficacy annular array tFUS system 100.

FIG. 1B illustrates achieving different focal depths using annular array 120, shown in System 100.

FIG. 2 shows an exemplary block diagram used in System 100 for computing acoustic data, which is then overlaid with MRI data and displayed on display 170.

FIG. 3 is an exemplary method 300 that uses an annular array tFUS system to achieve high efficacy.

FIG. 4A is an exemplary high-efficacy non-imaging matrix array tFUS system 400.

FIG. 4B shows an example operation of System 400 where the steered and focused stimulation point is off-axis.

FIG. 4C shows an example operation of System 400, where the System detects elements with poor or no acoustic contact.

FIG. 4D shows an example operation of System 400, where the System performs skull aberration measurements.

FIG. 5 is an exemplary method 500 that uses a non-imaging matrix array tFUS system to achieve high efficacy.

FIG. 6 is an exemplary high-efficacy annular array tFUS system 600 integrated with an EEG (Electroencephalography) system.

FIG. 7 is an exemplary method 700 that uses a high-efficacy integrated annular array tFUS system and EEG system.

### DETAILED DESCRIPTION

Systems and methods using non-imaging transcranial focused ultrasound (tFUS) systems are described. Non-imaging annular and matrix probes with low element counts are used in the systems. In an embodiment, an infrared-based system is used to gather the ultrasound's position information on the scalp. The position information is used to simulate the spatial distribution of the ultrasound field of the ultrasound probes. The simulation output is overlaid with pre-procedure MRI data and displayed to a clinician. Measurements on the MRI data are used to compensate the beam formation for the acoustic behavior of the skull so that a desirable tFUS focal region is achieved. In a different embodiment, an optical system is used for gathering positional information.

Annular array probes (“annular arrays”) consist of several concentric acoustic elements and are known to form well-focused beams even with small element counts that straightforward electronics can drive. Two-dimensional rectangular arrays (phased array probes, “matrix arrays”) with small element counts can also be produced easily, so long as the number of elements is low enough to allow for traditional interconnection. Matrix imaging probes typically have far more elements than traditional cabling and interconnect can accommodate. However, low element count matrix arrays are useful in the specific case of ultrasonic brain stimulation.

This application discloses methods and systems for reliably using simple, low-frequency probe (probe arrays or probes) designs to deliver ultrasound to targeted brain anatomy. Examples of these are:
(i) An annular array with several rings—as few as four rings.
(ii) A low-element-count non-imaging array, such as a 12×12-element array.

In this application, (i) annular arrays and (ii) low-element-count non-imaging arrays collectively are referred to as “simple probes” to distinguish them from probes capable of imaging inside the skull. Simple probes may be used for tFUS treatment if some means other than ultrasound can be found to guide the stimulation beam.

Simple probes may be used for tFUS treatment if some mean other than ultrasound can guide the stimulation beam to the targeted treatment region. Annular array stimulation can be achieved by phasing drive signals applied to the array's rings so that the focus along its central axis is moved deeper or shallower to match the subject's anatomy. This is an improvement over single-element probes currently in use for tFUS. An embodiment of this invention involves adjusting the phasing to account for scalp and skull thickness variations.

With a non-imaging matrix array, pulse-echo data may be obtained from several beam directions since the array can steer. Knowing the geometry of the skull's inner surface at several locations helps measure the dimensions of the head. These measurements find use in adapting an average head model to the subject being treated. They also help with improving the accuracy of the non-ultrasound targeting system.

A transmit-receive US system can inform the clinician if a blood vessel is present along the path to the treatment area or beyond. This information can be displayed using, for example, color M-mode. The presence of blood vessels may cause the clinician to select a different path for ultrasound to the target for the treatment. With an annular array, wedge stand-offs can provide steering capability, offering options to the clinician.

In an embodiment, the systems disclosed verify the acoustic contact of the probes with the subject's scalp. A pattern recognizer in the system analyzes the non-beamformed channel data and determines if there is adequate contact between the probes and the scalp. Feedback is provided to the clinician using a notification, such as a message on a display, visual cues or audible alerts.

In an embodiment, an EEG (electroencephalogram) system is integrated to provide guidance for placement of the simple probes. Feedback from EEG system such as EEG frequency bands, event-related potentials, etc. are used to guide the placement and orientation of the simple probes.

In an embodiment, an optical system is integrated with the system. The optical system is used for gathering scalp or head shape and the adapting an average head MRI to estimate the target location. The optical system may consist of a camera (such as camera on a cellphone or smart phone), LiDAR, etc.

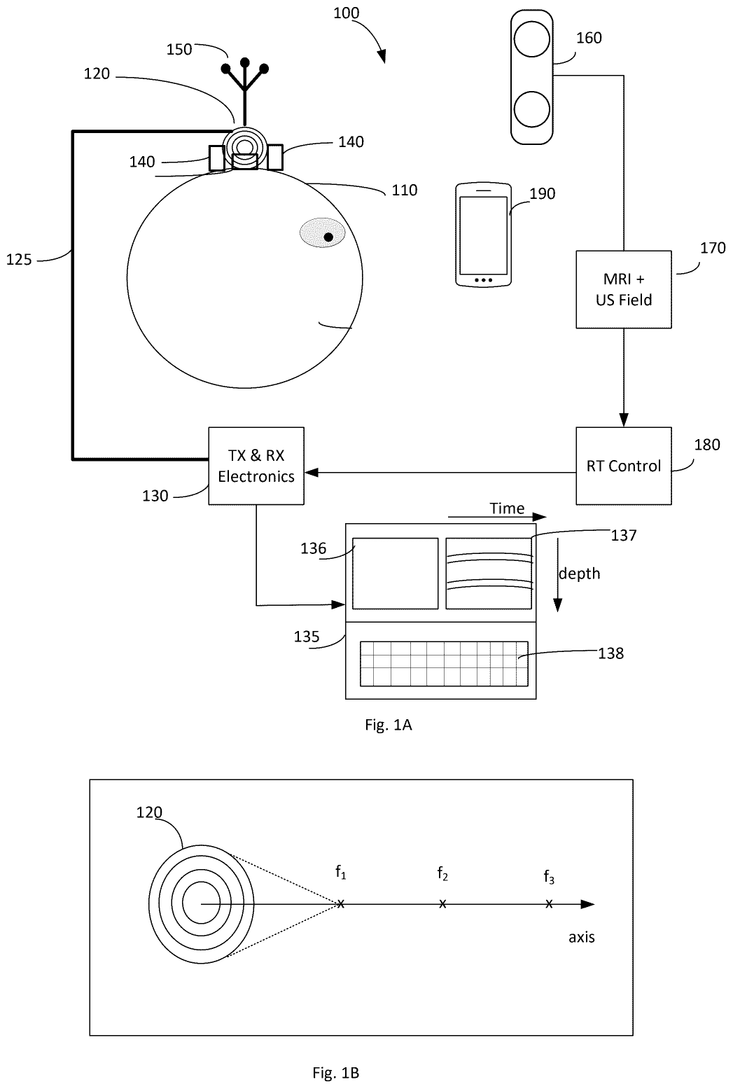

FIG. 1A is an exemplary high-efficacy annular array tFUS system 100. Referring to FIG. 1, an annular array 120 is placed on subject (or patient) 110's skull at a suitable location. Annular array 120 is depicted with four concentric annular elements (the central element is circular, while the others are rings). Other arrangements with a different number of annular rings can be used. Annular array stimulation can be achieved by phasing drive signals applied to the array's rings so that the focus along its central axis is moved deeper or shallower to match the subject's anatomy. Annular array probes provide excellent axial resolution with a small number of elements.

Attached to the array 120 are a set of infrared reflecting balls 150. System 100 includes two or more spaced infrared cameras 160, which use the infrared reflecting balls 150 to gather information on the position and orientation of the array 120. FIG. 1 shows a dual camera arrangement 160. System 100 uses the positional information gathered by camera 160 to simulate the ultrasound field the probe 120 will produce. It is overlayed on MRI (Magnetic resonance imaging) data and displayed (to a clinician) in display 170.

Before the ultrasound stimulation procedure starts, subject 110 can be placed in an MRI scanner to gather MRI scan data. The subject's 110 MRI scan data is loaded into System 100 from this pre-procedure MRI scan. This ultrasonic field overlay is accurate because it uses skull data from the MRI scan. Measurements on the MRI data are used to compensate the beam formation for the acoustic behavior of the skull so that a desirable tFUS focal region is achieved. The MRI scan data (consisting, for example, of T1 or T2 images) may be processed to provide estimates of acoustic properties such as density and sound speed, which are relevant to ultrasound propagation through the skull. For example, prior art AI techniques may be used to estimate the Hounsfield (CT) image from the MRI, which can be converted via well-known heuristics into density and sound speed data.

Beam formation coefficients are sent via the “RT (real-time) Control” block 180 to the electronics 130 (“TX electronics”), which creates the drive signals for the annuli in the array 120. The array's 120 geometry does not allow for complete correction of the skull aberration; however, errors in focal depth caused by the mean skull thickness and sound speed can be compensated for. RT Control 180 runs a phase adaptation algorithm that compensates for the geometry of the scalp and the skull. Control 180 selects beam formation coefficients based on the target location from the MRI data. In the annular array case, the ultrasonic beam is emitted broadside to the array, though the focus is variable. If the subject's anatomy is unsuitable for broadside access to the stimulation target, a wedge-shaped, acoustically transparent spacer may be used between the probe and the scalp.

Also shown in box 130 is “RX electronics,” which makes calculations allowing for a display of information captured along the center line of the array using a computer 135's display. An example, known in the art as “M (motion)-mode,” is illustrated (labeled 137). This is a two-dimensional image where the vertical dimension is the depth down the center line, and the horizontal dimension is slow time. Typically, the display scrolls to the left, indicating how tissue motion and blood flow vary over time. It is common to use an autocorrelation technique to the repetitive firings of the transmit-receive system to estimate blood flow speed, which may be encoded in the display using color. Computer 135 (with keyboard 138) shows a display (labeled 136) that includes skull measurements, such as information about any ventricular gap along the axis of the probe. An M-mode Doppler display 137 is also shown. In an embodiment, System 100 allows the clinician to be notified if a blood vessel is present along the path to the treatment area or beyond it. This information can be displayed using, for example, color M-mode 137. The presence of blood vessels may cause the clinician to select a different ultrasound path for the treatment. With the annular array, wedge stand-offs can provide this capability, essentially introducing a fixed steering angle to the beam.

The annular array 120 may be attached to a gimbal mount 140 fixed to the headset (not shown). A conformable coupling layer allows for accurate adjustment of the angle of the axial beam as it travels from the probe to the target. If the gimbal is motorized, Electronics 130 can control the gimbal mount 140 wirelessly or through a wired interface.

In a different embodiment, illustrated in FIG. 1A, optical guidance is used without a stereoscopic camera 160 or orienting balls 150 to guide the placement and orientation of probe 120. This may be a cell phone 190 with a camera, LiDAR, or other optical device providing data about the shape of the subject's 110 head. These data deform an average head MRI, such as the MNI (Montreal Neurological Institute and Hospital) dataset, to estimate the target's location in the brain (e.g., amygdala or posterior cingulate cortex) and its relationship to the probe's 120 position and orientation. In the annular array system using optical guidance, the computations may be done on the phone's 190 CPU and GPU, and the probe 120 assembly may communicate wirelessly. Electronics may be provided with the probe 120 assembly on the subject's 110 head containing transmitter ICs. The waveforms may be generated by algorithms encoded in an FPGA feeding the transmitter ICs.

In an embodiment, the annular array is capable of single-line receive capability. This allows for measurement of the back and front of the skull along the central line. This is helpful in further adapting the average head to the specifics of the patient's head anatomy. Further information to improve the adaptation can be found from the single-line pulse-echo data if a ventricle is within the beam path. Ventricles are fluid-filled and, as such, do not scatter ultrasound. Thus, they are easy to detect.

FIG. 1B illustrates achieving different focal depths using annular array 120, shown in System 100. Annular array stimulation can be achieved by phasing drive signals applied to the array's rings so that the focus along its central axis is moved deeper or shallower to match the subject's anatomy. FIG. 1B shows the effect of drive voltages applied to the annular array 120 with different phases, achieving different focal depths, f1, f2, and f3.

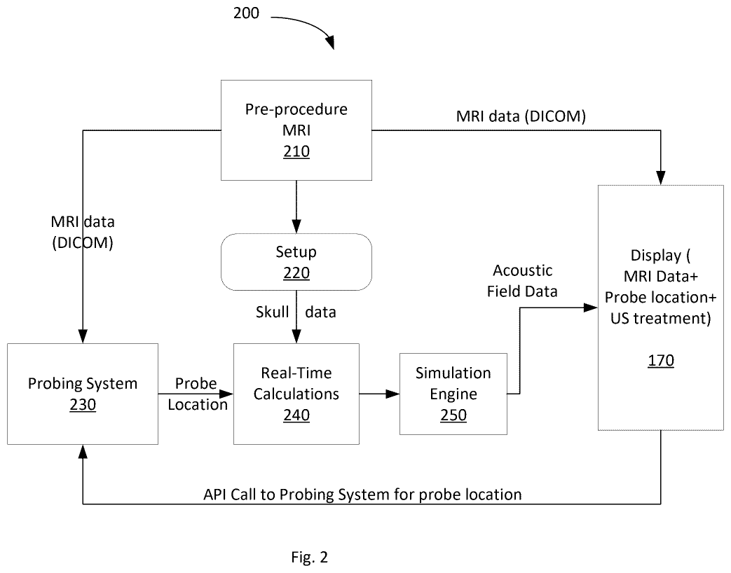

FIG. 2 shows an exemplary block diagram used in System 100 for computing acoustic data, which is then overlaid with MRI data and displayed on display 170. The figure illustrates how accurate acoustic field data can be computed and overlaid on the pre-procedure MRI. This enables a clinician to understand where the ultrasonic beam will travel on its way to the stimulation target before starting the treatment. Pre-procedure MRI data in DICOM (Digital Imaging and Communications in Medicine) format is loaded into System 100 and stored (at Pre-procedure MRI 210). The MRI data is used by geometry system 230 (also referred to as “neuronavigation systems”, which can relate the position and orientation of the transducer to the coordinate system of a pre-procedure MRI) and display 170. The MRI data may be converted into spatial distributions of Speed of Sound (SOS) and density values, and skull contour information is created in Setup 220 and sent to real-time calculation 240. Once the probe appears well-positioned to the clinician, the system requests the current location, orientation, etc., of probe 120 using the geometry system 230. The geometry system 230 comprises the infrared camera 160 and infrared reflecting balls 150 to compute the probe 120's location, etc. Geometry system 230 sends the probe location and pose data to real-time calculations 240. The scene to simulate is generated from the skull data and the probe 120 position, angular pose, and element delays applied to the probe 120. The potential arrangement for stimulation to simulate is sent to a computation engine 250, which runs an acoustic simulator [such as a ray-tracing code, pseudo-spectral code, or a finite element code] to create a beam pattern considering all the arrangement's features. The composite data set, including MRI data, probe position/pose, and ultrasound beam (ultrasound treatment plan), is displayed on display 170, where the clinician can navigate the view in 3D. If the prospective simulation is not deemed adequate by the clinician, the probe may be repositioned. If probe 120 is repositioned, the beam is updated on the screen to reflect the effect of its new position/pose. The clinician may also alter the location of the stimulation target in 3D by navigating to the desired structure in the MRI data and indicating the location with a mouse click.

### Method for Simple Probe Annular Array

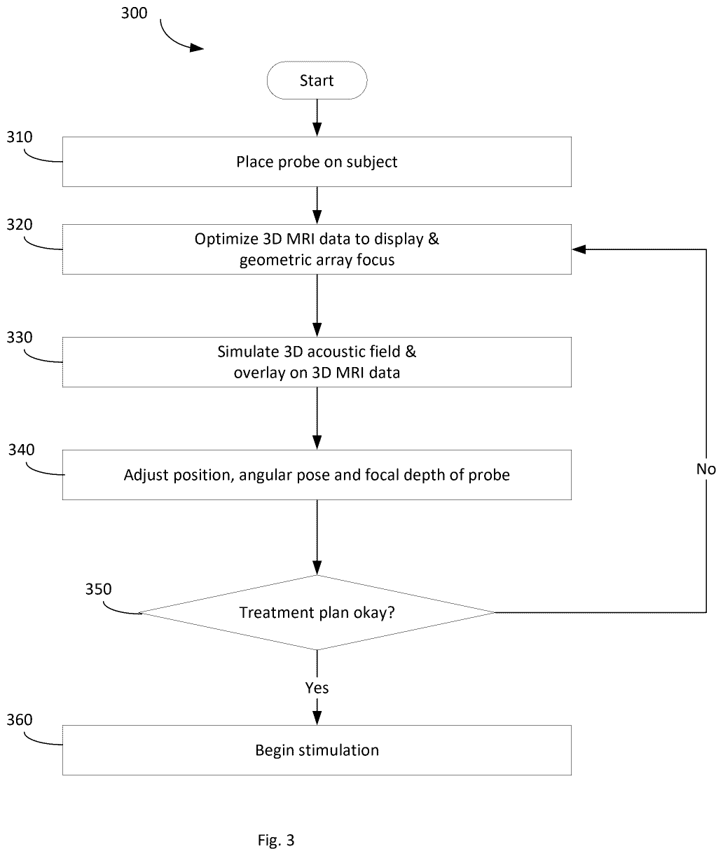

FIG. 3 is an exemplary method 300 that uses an annular array tFUS system to achieve high efficacy. The method 300 can be performed using System 100. At operation 310, the probe (or annular array) 120 is placed on subject 110's head so that, geometrically, the focus of the array is close to the target anatomy. A clinician may use the geometry system and the gimbal mount 140 to optimize the placement of probe 120.

At operation 320, the subject's 110 MRI data is loaded into System 100 and displayed on display 170. The MRI data is converted into Speed of Sound (SOS) and density, and skull contour information is created. A clinician scrolls through the subject's MRI data to optimize the display of the relevant part of the brain anatomy and the geometric array focus. The geometry system provides the current location, orientation, etc. of the probe 120. System 100 uses the probe position, angular pose, and element delays applied to probe 120 with the target location of the skull data to create a scene for simulation.

At operation 330, based on the MRI and the position of the array 120, system 100 simulates the 3D acoustic field, which is then overlaid on the 3D MRI data to be displayed on display 170. The “scene to simulate” generated at operation 320 is simulated by system 100 to generate the US beam pattern. A composite 3D image of the MRI data, US beam pattern, and probe 120's location & pose is generated.

At operation 340, the clinician adjusts the probe's position, angular pose, and focal depth. This is necessary because the annular array cannot steer. The position and angular pose can be adjusted using gimbal mount 140. If necessary, the clinician manually adjusts the position of the array 120. System may notify the clinician of any blood vessels present along the path of the treatment or beyond it. The focal depth is adjusted using phasing drive signals applied to the array's 120 rings or elements. Adjusting the phasing drive signal compensates for the geometry of the scalp and skull.

At operation 350, the clinician views the 3D acoustic field overlaid on the 3D MRI data on display 170 and determines if the current treatment plan is okay. If the clinician is unsatisfied with the current treatment plan, method 300 returns to operation 320. If the clinician is satisfied, method 300 proceeds to operation 360.

At operation 360, the array's 120 position and orientation are fixed, and the stimulation is started.

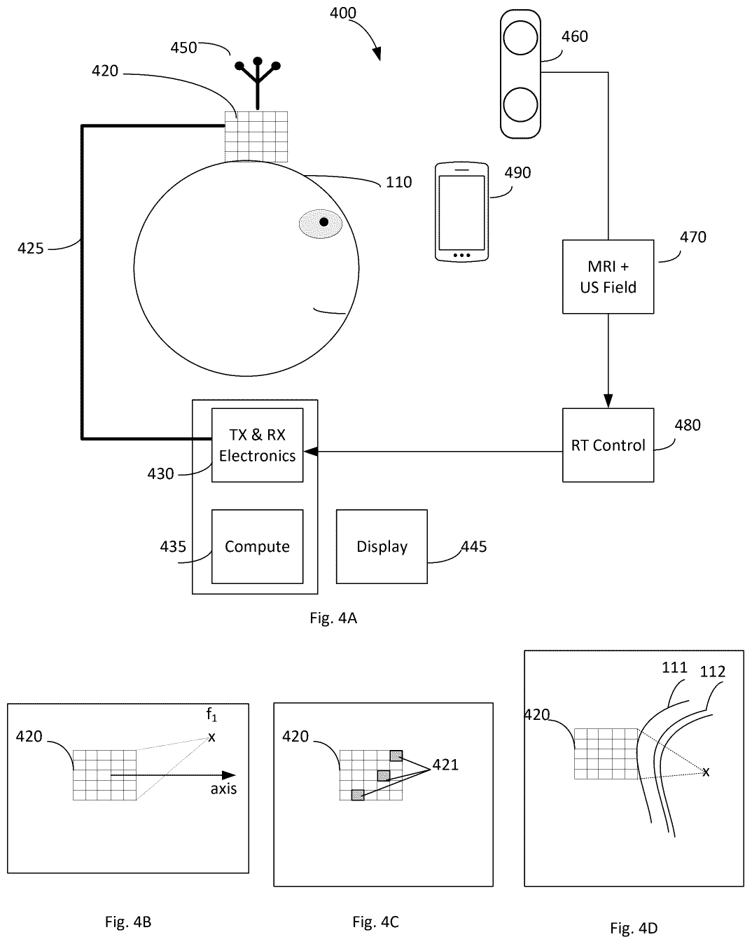

FIG. 4A is an exemplary high-efficacy non-imaging matrix array tFUS system 400. System 400 is similar to System 100 with a modification in which a non-imaging, rectangular matrix array replaces the annular array 120. Low element count matrix arrays, which would not be considered for diagnostic imaging systems due to low image quality capability, can function effectively in this application. In System 400, improvements in accuracy are possible beyond the simple depth correction. System 400 can be used in four modes:

tFUS stimulation, where this type of probe allows for beam steering in addition to focusing.

Acoustic contact verification, in which the individual elements transmit and receive, and the non-beamformed channel data is fed to a pattern recognizer to determine whether the acoustic path to the scalp is intact. This is extremely important because the loss of acoustic contact during the stimulation protocol is expected and dramatically decreases treatment efficacy by distorting the stimulation beam.

Two-dimensional mapping of the acoustic response of the scalp and skull under each probe element, from which aberration correction coefficients may be computed.

Measurement of the distance between the probe and the inner surface of the far side of the skull to better adapt an average head model to the subject's anatomy. This distance measurement may usefully be performed at a number of beam steering angles.

Referring to FIG. 4, a non-imaging matrix array 420 is placed on subject (or patient) 110's skull at an appropriate location. Array 420 is positioned at a favorable location on the head, providing optimal mechanical stability. In FIG. 4, array 420 is shown for clarity with its elements in the diagram plane; in practice, its elements would be positioned to contact the skull. Electronic focusing and steering with aberration correction are used to obtain an acoustic focus of the correct shape and depth to treat the target anatomy.

Array 420 may have a set of infrared reflecting balls 450. System 400 includes an infrared camera 460, which can use the infrared reflecting balls 450 to gather information on the position and orientation of the array 420. This is a clinically verified technique termed “neuronavigation.” FIG. 4 shows a dual camera 460. System 400 uses the probe position information gathered by camera 460 to simulate the ultrasound field the probe 420 will produce. It is overlayed with MRI (Magnetic resonance imaging) data to form a composite 3D image and displayed (to a clinician) in display 470. In another example, the subject's 110 MRI scan data is loaded into System 400 from an MRI scan performed beforehand. This ultrasonic field overlay is accurate because it uses skull data from the MRI scan. Measurements on the MRI data characterizing the scalp and skull anatomy are used to compensate the beam formation for the acoustic behavior of the skull so that a desirable tFUS focal region is achieved. Alternatively, subject 110 and System 400 can be placed in an MRI scanner to gather MRI scan data to verify that the target was correctly stimulated.

Beam formation coefficients and aberration correction data for each probe element are sent via the “RT (real-time) Control” block 480 to the electronics 430 (“TX electronics”), which creates the signals for the elements in the array 420. Control 480 selects beam formation coefficients and skull aberration correction based on the patient's anatomy characterized from the MRI data. The operation of Control 480 may also be informed by measurements of the patient's scalp and skull anatomy obtained by transmitting and receiving the elements of array 420.

Attached to the TX & RX electronics 430 is the compute block 435. Compute block 435 controls System 400 operations in the three modes. Also shown in the same box 430 is “RX electronics,” which allows a display of information captured from the array using a computer 445's display. For instance, M-mode Doppler data and other skull measurements can be displayed.

FIG. 4B shows an example operation of System 400 where the steered and focused stimulation point is off-axis. Referring to FIG. 4B, array 420 can steer and focus on a point f1 that is off-axis. The ultrasonic beam direction and focus can be steered electronically by varying the excitation delay of individual probe elements or groups of elements in the array 420. Similar to System 100, System 400 can achieve different focal depths. In addition, the direction of the focal point can also be steered.

System 400 can detect whether individual elements have a good acoustic path or contact with the scalp in an embodiment. This is done by feeding the non-beamformed data to a pattern recognizer operating in the compute block 435. FIG. 4C shows an example operation of System 400, where the System detects elements with poor or no acoustic contact. Referring to FIG. 4C, shaded elements 421 have poor or no acoustic contact with the scalp.

In another embodiment, skull aberration measurements are performed by System 400 by two-dimensional mapping of the acoustic response of the scalp and skull under each probe element. FIG. 4D shows an example operation of System 400, where the System performs skull aberration measurements. Again, array 420 is rotated for clarity in FIG. 4D.

In a different embodiment, optical guidance is used. This may be a cell phone 490 with a camera, LiDAR, or other optical device providing data about the shape of the subject's 110 head. These data may be used to adapt an average head MRI, such as the MNI (Montreal Neurological Institute and Hospital) dataset, to estimate the target's location in the brain (e.g., amygdala or posterior cingulate cortex) and its relationship to probe 420's position. In the annular array system using optical guidance, the computations may be done on the phone's 490 CPU and GPU, and the probe 420 assembly may communicate wirelessly. The electronics with the probe 420 assembly on the subject's 410 head contain transmitter ICs. The waveforms are generated by algorithms encoded in an FPGA feeding the transmitter ICs.

### Method for Simple Probe Matrix Array

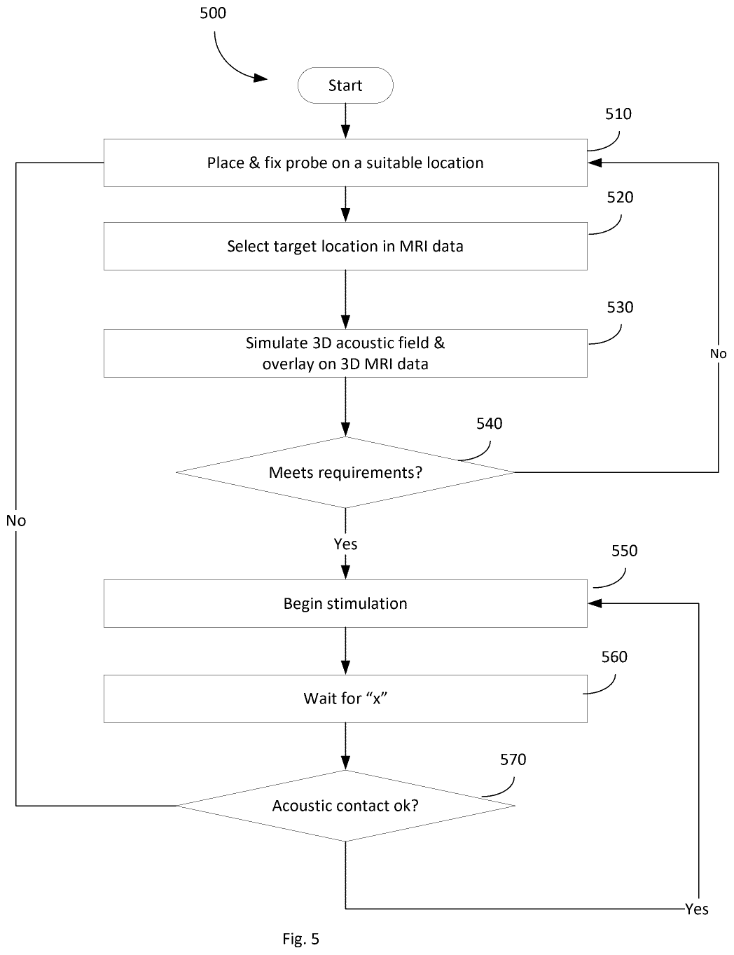

FIG. 5 is an exemplary method 500 that uses a non-imaging matrix array tFUS system to achieve high efficacy. Method 500 may be performed using System 400. At operation 510, a clinician analyzes the subject's head and selects the most stable position for probe 420. The probe is fixed there with a mild adhesive. This prevents any unwanted movement of probe 420.

At operation 520, System 400 is loaded with the subject's MRI data. The clinician scrolls through the subject's MRI data and selects the target position.

At operation 530, System 400 overlays the stimulation field on the MRI data. The stimulation field is accurate because it considers ultrasonic pulse-echo measurements along with the MRI data, which characterize the scalp and skull anatomy details.

At operation 540, the clinician checks if the planned stimulation meets the treatment requirements. If requirements are not met, method 300 returns to operation 510. If requirements are met, method 500 proceeds to operation 550.

At operation 550, the clinician begins stimulation. At operation 560, method 500 waits for a certain period (e.g., 1 second). At operation 570, method 500 checks to see if the acoustic contact of the active elements of the array 420 are okay. If it is determined contact is okay, stimulation is continued (Operation 550). If the contact is not okay, method 500 returns to operation 510. For instance, a pattern recognizer analyzes the non-beamformed channel data to determine if there is adequate contact between the probes 420 and the subject's scalp. Feedback is provided to the clinician that adequate contact between the probes and subject's scalp is not there. Feedback is provided to the clinician using display 445, visual cues (error LEDs or flashing LEDs, etc.), or audible alerts.

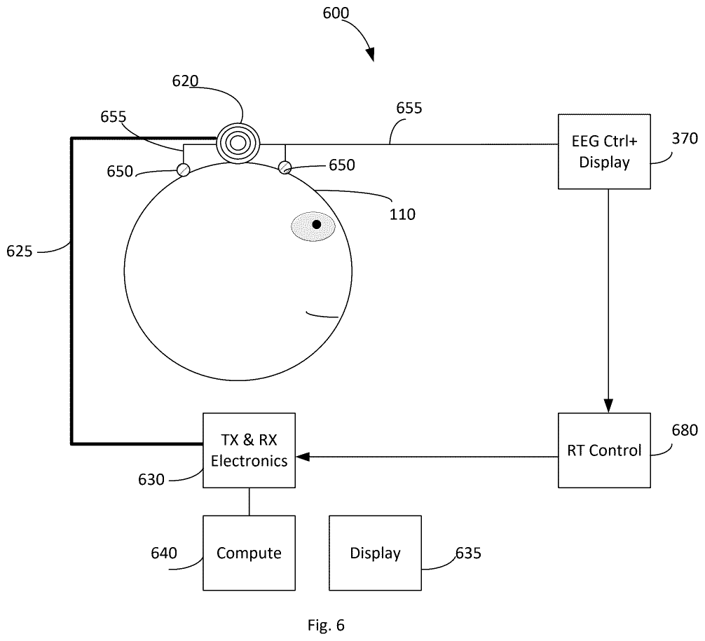

FIG. 6 is an exemplary high-efficacy annular array tFUS system 600 integrated with an EEG system. System 600 is similar to System 100 but uses EEG for guidance. Referring to FIG. 6, an annular array 620 is placed on subject (or patient) 110's skull at a suitable location. Annular array 620 is depicted with four concentric annular elements. Other arrangements with a different number of annular rings can be used. (concentric rings). Attached to the subject 110's skull is an EEG headset. EEG electrodes 650 are attached to subject 110's skull at appropriate locations. The figure only shows two EEG electrodes to simplify the figure. EEG electrodes are connected to an “EEG CTRL+Display” 370 using appropriate wires 655. EEG Ctrl 370 controls the operation of the EEG and displays EEG data after the appropriate filtering (Low-pass, High-pass, Notch-pass, etc.). Special considerations are required to ensure no mutual interference between the EEG and the annular probe systems.

EEG control analyzes the EEG data for EEG biomarkers (a quantitative measure derived from EEG, e.g., dominant frequency in the delta frequency range). The absence or presence of specific biomarkers can indicate the efficacy of the ultrasound stimulation. Feedback can be event-related potentials. Based on the feedback, the clinician adjusts the location or pose of the annular array 620. Similar to System 100, annular array 620 is attached to a gimbal mount (not shown) fixed to the headset (not shown). This allows for accurate adjustment of the angle of the axial beam as it travels from the probe to the target. TX & RX Electronics 630 can control the gimbal mount wirelessly or through a wired interface. Beam formation coefficients are sent via the “RT (real-time) Control” block 680 to the electronics 630 (“TX electronics”), which creates the drive signals for the annuli in the array 120.

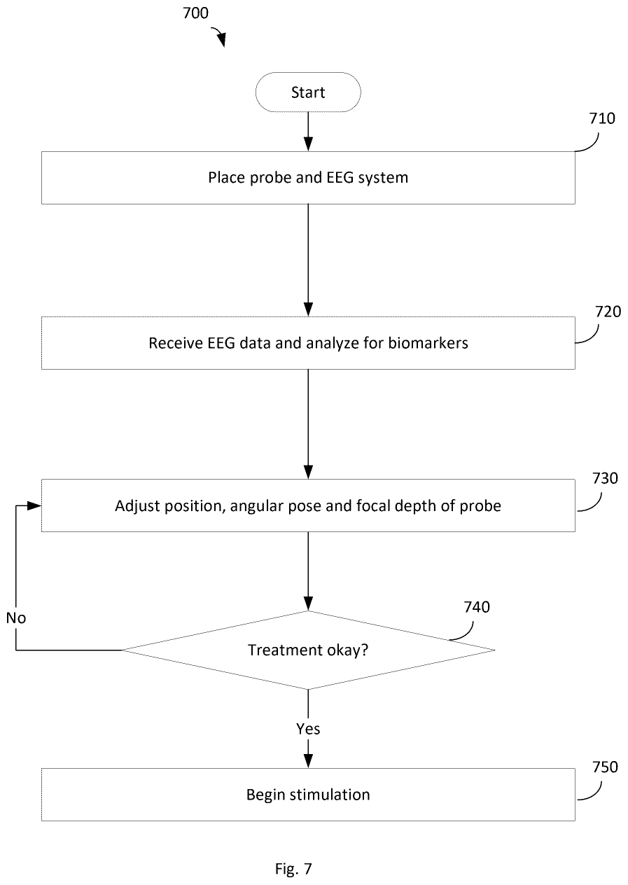

FIG. 7 is an exemplary method 700 that uses an integrated high-efficacy annular array tFUS system and EEG system. Method 700 can be performed using System 600. At operation 710, the annular probe 620 and EEG electrodes 650 are placed on the subject scalp at appropriate locations. Special care is taken to ensure that there is no mutual interference between the systems.

At operation 720, the target anatomy is ultrasonically stimulated. EEG data is analyzed for biomarkers. The absence or presence of specific biomarkers can indicate the efficacy of the US stimulation.

At operation 730, the clinician adjusts the probe's position, pose, and focal depth based on feedback received from operation 720.

At operation 740, the clinician checks to see if the treatment works as expected. If it is working correctly, the following operation is operation 750. If the treatment is not working correctly, the probe position, pose, and depth are adjusted at operation 730.

What is claimed is:

1. A system comprising:
a probe array configured to emit transcranial focused ultrasound;
a gimbal system coupled to the probe array;
at least one infrared camera;
a reflecting device positioned proximate to the probe array; and
a computing device executing an application that causes the at least one computing device to at least:
cause the at least one infrared camera to gather positional information from the reflecting device;
simulate an ultrasound field produced by the probe array based on the positional information; and
in response to determining a position and orientation of the probe array based on the simulated ultrasound field, initiating ultrasound stimulation via the probe array.

2. The system of claim 1, wherein the probe array comprises an annular probe array or a rectangular matrix array.

3. The system of claim 1, where the probe array comprises a low element-count matrix array.

4. The system of claim 1, wherein the application further causes the computing device to at least:
receive MRI data associated with a patient;
overlay the simulated ultrasound field onto the MRI data; and
generate a scene associated with the patient based on the overlaid simulated ultrasound field.

5. The system of claim 4, wherein the application further causes the computing device to at least display the scene in a user interface.

6. The system of claim 1, wherein the application further causes the computing device to at least generate beam formation coefficients corresponding to drive signals for the probe array, the beam formation coefficients based upon skull thickness or skull aberrations of a patient.

7. The system of claim 1, wherein the application further causes the computing device to cause the gimbal system to adjust positioning of the probe array.

8. The system of claim 1, wherein the reflecting device comprises a plurality of reflecting balls positioned proximate to the probe array.

9. The system of claim 1, wherein the at least one infrared camera gathers positional information from the reflecting device based on infrared reflections from the reflecting device.

10. The system of claim 1, wherein the application causes the computing device to compensate the ultrasound stimulation based on a phase adaptation algorithm compensating for at least one of scalp geometry or skull geometry.

11. The system of claim 10, wherein the at least one of scalp geometry or skull geometry are based on MRI imaging data.

12. The system of claim 1, wherein the system identifies a blood vessel in the simulated ultrasound field and generates a notification in response to identifying the blood vessel.

13. The system of claim 1, further comprising an optical guidance system, the optical guidance system measuring data about the shape of a scalp, wherein the application executed by computing device estimates a target position and orientation of the probe array based on the data provided by the optical guidance system.

14. The system of claim 13, wherein the optical guidance system comprises at least one of a camera, camera on a smartphone, or a LIDAR device.

15. The system of claim 1, further comprising an EEG system, wherein EEG biomarkers or event-related potentials measured by the EEG system are used by the application executed by the computing device to calculate a target position and orientation of the probe array.

16. The system of claim 3, wherein the application verifies acoustic contact between probes of the probe array and a scalp and generates at least one notification on acoustic contact verification.

17. The system of claim 16, wherein the application analyzes non-beamformed probe data from the probe array to determine if there is adequate contact between the probe array and a scalp.

18. A method comprising:
obtaining magnetic resonance imaging (MRI) data associated with a patient;
simulating, based on the MRI data, and positional data associated with a probe array configured to emit transcranial focused ultrasound, an acoustic field associated ultrasound emissions of the probe array;
adjusting, based on the acoustic field simulation, a position of the probe array; and
stimulating the patient using the probe array from the adjusted position of the probe array.

19. The method of claim 12, wherein the probe array comprises an annular probe array or a rectangular matrix array.

20. The method of claim 12, where the probe array comprises a low element-count matrix array.

21. The method of claim 12, further comprising displaying the simulated acoustic field overlaid onto the MRI data in a user interface.

22. The method of claim 12, further comprising generating beam formation coefficients corresponding to drive signals for the probe array, the beam formation coefficients based upon skull thickness or skull aberrations of a patient.

23. The method of claim 12, further comprising adjusting positioning of the probe array using a gimbal device.

24. The method of claim 12, further comprising determining the positional data associated with the probe array based upon data obtained by at least one infrared camera.

25. The method of claim 18, wherein the at least one infrared camera obtains the positional data based on reflections from a reflecting device mounted proximate to the probe array.

26. The method of claim 19, wherein the reflecting device comprises a plurality of reflecting balls positioned proximate to the probe array.

27. The method of claim 12, further comprising compensating the ultrasound stimulation based on a phase adaptation algorithm compensating for at least one of scalp geometry or skull geometry.

28. The method of claim 12, wherein the at least one of scalp geometry or skull geometry are based on the MRI data.

29. The method of claim 18, further comprising identifying a blood vessel in the simulated ultrasound field and generates a notification in response to identifying the blood vessel.

30. The method of claim 18, further comprising measuring, via an optical guidance system, data about the shape of a scalp, wherein the application executed by computing device estimates a target position and orientation of the probe array based on the data provided by the optical guidance system.

31. The method of claim 18, wherein EEG biomarkers or event-related potentials measured by an EEG system are used to calculate a target position and orientation of the probe array.

32. The method of claim 18, further comprising verifying acoustic contact between probes of the probe array and a scalp and generates at least one notification on acoustic contact verification.

33. The method of claim 32, further comprising analyzing non-beamformed probe data from the probe array to determine if there is adequate contact between the probe array and a scalp.
